(19) United States
(12) Patent Application Publication
(10) Pub. No.: US 2009/0118584 A1
(43) Pub. Date: May 7, 2009

(54) INSERTION TUBE FOR ENDOSCOPE AND ENDOSCOPE

(75) Inventors: Shigeru KOSUKI, Hachioji-shi (JP); Koji TAKAMURA, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/256,022

(22) Filed: Oct. 22, 2008

(30) Foreign Application Priority Data
Oct. 24, 2007 (JP) 2007-276754

(51) Int. Cl. A61B 1/008

(52) U.S. Cl. 600/140

(57) ABSTRACT

An insertion tube for an endoscope includes a braid tube, the braid tube includes fine line portions arranged in a braided manner and intersecting portions where the fine line portions intersect with each other, and the fine line portions is restricted with each other in the intersecting portions.

### CROSS-REFERENCE TO RELATED APPLICATIONS

This application is based upon and claims the benefit of priority from prior Japanese Patent Application No. 2007-276754, filed Oct. 24, 2007, the entire contents of which are incorporated herein by reference.

### BACKGROUND OF THE INVENTION

1. Field of the Invention

The present invention relates to an insertion tube for an endoscope and an endoscope.

2. Description of the Related Art

An endoscope includes an elongated insertion portion to be inserted into a body cavity. In the insertion portion, various built-in components are inserted through an insertion tube for the endoscope. In order to secure the sufficient insertion and operation performance of the insertion portion, it is required for the insertion tube for the endoscope that sufficient softness, flexibility, resiliency and followability. In order to satisfy such requirements, the insertion tube for the endoscope is usually formed by successively superposing a spiral tube formed of a wound strip-like member, a braid tube formed of braided fine line members and an outer tube made of a resin from an inner side to an outer side.

In Jpn. Pat. Appln. KOKAI Publication No. 5-95898, a flexible tube for forming an accessory insertion channel of the endoscope is disclosed. The flexible tube includes the three-layer structure similar to that of the insertion tube for the endoscope. Furthermore, in order to improve strength, the fine line members of the braid tube are welded and fixed to each other in intersecting portions thereof.

In Jpn. Pat. Appln. KOKAI Publication No. 2006-61204, the insertion tube for the endoscope is disclosed. In the insertion tube for the endoscope, in order to improve the resiliency, a linear member having superelasticity is interposed between the outer tube and the braid tube, and the linear member spirally or linearly extends in the longitudinal direction of the braid tube.

### BRIEF SUMMARY OF THE INVENTION

In an aspect of the present invention, an insertion tube for an endoscope includes a braid tube, the braid tube includes fine line portions arranged in a braided manner and intersecting portions where the fine line portions intersect with each other, and the fine line portions is restricted with each other in the intersecting portions.

In another aspect of the present invention, an endoscope includes an insertion tube for the endoscope, the insertion tube for the endoscope includes a braid tube, the braid tube includes fine line portions arranged in a braided manner and intersecting portions where the fine line portions intersect with each other, and the fine line portions are restricted with each other in the intersecting portions.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a side view showing an endoscope of a first embodiment of the present invention;

FIG. 2 is a longitudinal sectional view showing an insertion tube for the endoscope of the first embodiment of the present invention;

FIG. 3 is a schematic side view showing a braid tube of the first embodiment of the present invention;

FIG. 4 is an exploded view showing a braid tube of a first modification example of the first embodiment of the present invention;

FIG. 5 is an exploded view showing a braid tube of a second modification example of the first embodiment of the present invention;

FIG. 6 is a schematic diagram showing a braid tube and a linear member of a second embodiment of the present invention;

FIG. 7 is a schematic diagram showing a braid tube and a linear member of a first modification example of the second embodiment of the present invention;

FIG. 8 is a schematic diagram showing a braid tube and a linear member of a second modification example of the second embodiment of the present invention;

FIG. 9 is a schematic diagram showing a braid tube and a linear member of a third embodiment of the present invention;

FIG. 10 is a schematic diagram showing a braid tube and a linear member of a first modification example of the third embodiment of the present invention;

FIG. 11 is a schematic diagram showing a braid tube and a linear member of a second modification example of the third embodiment of the present invention;

FIG. 12 is a longitudinal sectional view showing a braid tube of a fourth embodiment of the present invention;

FIG. 13 is a longitudinal sectional view showing a braid tube of a first modification example of the fourth embodiment of the present invention;

FIG. 14 is a longitudinal sectional view showing a braid tube of a second modification example of the fourth embodiment of the present invention;

FIG. 15 is a longitudinal sectional view showing an insertion tube for an endoscope of a first reference embodiment of the present invention;

FIG. 16 is a longitudinal sectional view showing a spiral tube of a second reference embodiment of the present invention;

FIG. 17 is a longitudinal sectional view showing a spiral tube of a third reference embodiment of the present invention; and

FIG. 18 is a longitudinal sectional view showing a spiral tube of a fourth reference embodiment of the present invention.

### DETAILED DESCRIPTION OF THE INVENTION

Embodiments of the present invention will hereinafter be described with reference to the drawings.

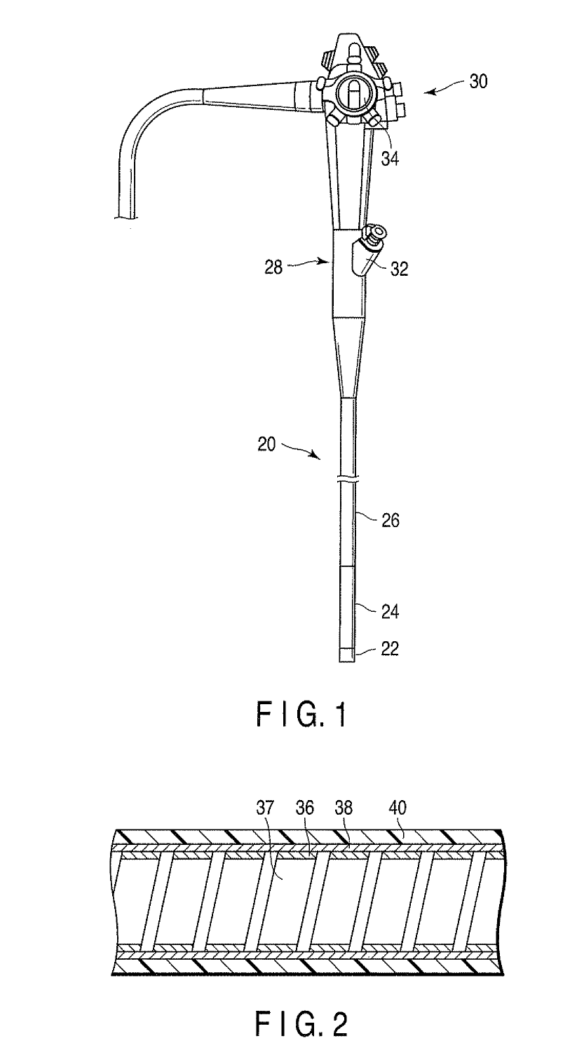

FIGS. 1 to 3 show a first embodiment of the present invention.

As shown in FIG. 1, an endoscope includes an elongated insertion portion 20 to be inserted into a body cavity. In the insertion portion 20, a distal end rigid portion 22 having rigidity, a bending portion 24 to be operated to be bent and an insertion tube portion 26 elongated and having flexibility are arranged from a distal end side to a proximal end side. In the insertion tube portion 26, various built-in components for illumination, observation, air/water supply, accessory insertion, the bending operation of the bending portion 24 and the like are inserted through an insertion tube for the endoscope. An operation portion 28 to be held and operated by an operator is connected to the proximal end of the insertion portion 20. The operation portion 28 is provided with various switches 30 for operating the illumination, image pickup, the air/water supply and the like, an accessory insertion connecter 32 for inserting an accessory, a bending operation knob 34 to operate the bending portion 24 to be bend and the like.

The insertion tube for the endoscope will be described with reference to FIG. 2.

In the insertion tube for the endoscope, a spiral tube 36, a braid tube 38 and an outer tube 40 are successively superimposed from an inner side to an outer side. The spiral tube 36 is formed by winding a strip-like member 37 made of a metal or a resin in a spiral shape. The braid tube 38 is formed by braiding a metal fine line in a tubular shape. The outer tube 40 is made of a resin.

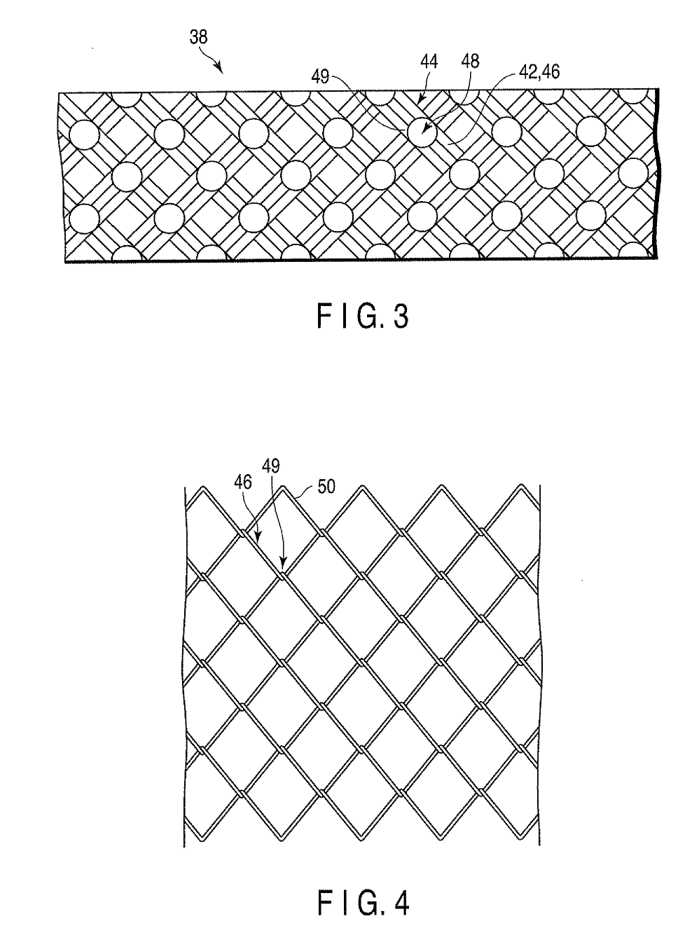

The braid tube 38 will be described in detail with reference to FIG. 3.

In the braid tube 38 of the present embodiment, a metal fine line 42 made of a stainless steel is used. A fine line bundle 44 is formed of the metal fine lines 42, and the braid tube 38 is formed by weaving the fine line bundles 44 through plain weave into a tubular shape. Therefore, the metal fine lines 42 as fine line members form fine line portions 46 arranged in a braided manner. In FIG. 3, as schematically shown by circular indicator, the fine line bundles 44 are welded and restricted to each other in intersecting regions 48 of the fine line bundles 44. That is, the fine line portions 46 formed of the metal fine lines 42 are welded and restricted to each other in intersecting portions 49 of the fine line portions 46.

In the insertion tube for the endoscope of the present embodiment, the metal fine lines 42 of the braid tube 38 are welded and restricted to each other in the intersecting portions of the metal fine lines 42. Therefore, the resiliency of the braid tube 38 and so the insertion tube for the endoscope is improved.

In the above embodiment, the fine line bundles 44 are welded in the respective intersecting regions 48 of the fine line bundles 44, but welding portions may appropriately be selected from a number of the intersecting regions 48 in accordance with desired resiliency. Moreover, the welding is used in restricting the metal fine lines 42 to each other in the intersecting portions 49 of metal fine lines 42, but bonding, brazing, soldering or the like may be used.

FIG. 4 shows a first modification example of the first embodiment of the present invention.

The braid tube 38 of the present modification example is chain link line netting. That is, the braid tube 38 is formed by processing metal lines 50 through bending processing into mountain shapes at constant pitches and entangling metal lines 50 with each other so that parallelogram meshes are formed and into a tubular shape. The metal lines 50 forming the fine line portions 46 are entangled and restricted with each other in the intersecting portions 49 of the metal lines 50.

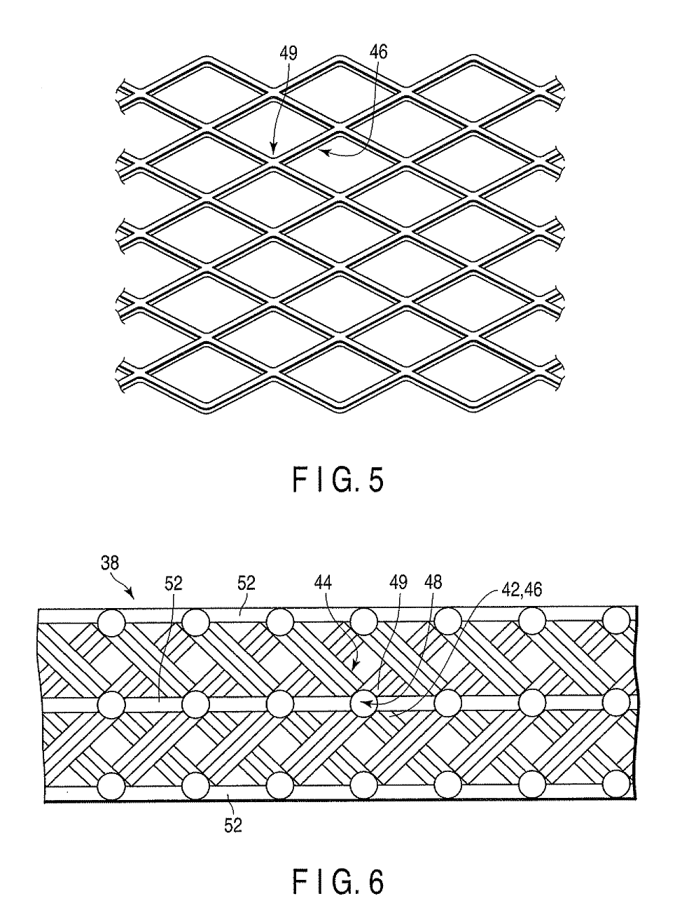

FIG. 5 shows a second modification example of the first embodiment of the present invention.

The braid tube 38 of the present modification example is an expanded metal. That is, the braid tube 38 is formed by making fine and alternate cuts in a metal thin plate, pulling the metal thin plate to expand into a wire netting shape, joining both side portions of the metal thin plate with each other into a tubular shape. The fine line portions 46 of the expanded metal are coupled and restricted with each other in the intersecting portions 49.

FIG. 6 shows a second embodiment of the present invention.

In the present embodiment, in order to improve the resiliency of the insertion tube for the endoscope, linear members 52 having superelasticity are used. Here, the superelasticity is a characteristic that disappearance of distortion and return to an original shape occur on removal of the external force even after application of an external force to such an extent to bring about plastic distortion. Specifically, examples of a superelastic alloy having the superelasticity include a nickel-titanium alloy, a nickel-titanium-iron alloy, a nickel-titanium-chromium alloy, and a nickel-titanium-copper-chromium alloy. Such superelastic alloys has characteristics that the return to an original shape occur even when large distortion of about 8% is generated, a yield stress is comparatively high, a distortion at a yield point is large and may be in excess of 1%, and a coefficient elasticity is small, is i.e. 6000 to 8000 kgf/mm².

The four linear members 52 extend along the outer peripheral surface of the braid tube 38 in the longitudinal direction of the braid tube 38, and the four linear members 52 are arranged at an equal interval, that is, an interval of 90° in the peripheral direction of the braid tube 38. Moreover, in the drawing, as schematically shown by the circular indicator, both the fine line bundles 44 are welded and restricted with each other and, in addition, the linear member 52 is welded and restricted with the braid tube 38 in each intersecting region 48 through which the linear member 52 pass among the intersecting regions 48 of the fine line bundles 44 of the braid tube 38. That is, the linear members 52 is welded and restricted with the braid tube 38 in the intersecting portions 49 of the fine line portions 46 formed of the metal fine lines 42.

In the insertion tube for the endoscope of the present embodiment, the linear members 52 having the superelasticity extend along the outer peripheral surface of the braid tube 38 in the longitudinal direction, and are welded and restricted with the braid tube 38 in the intersecting regions 48 of the fine line bundles 44 of the braid tube 38, so that the resiliency of the insertion tube for the endoscope is further improved.

Moreover, it is difficult to deform and process the linear members 52 having the superelasticity. In manufacture of the insertion tube for the endoscope, the linear members 52 may simply be arranged along the outer peripheral surface of the braid tube 38 and it is unnecessary to process the linear members 52. Moreover, it is possible to perform the welding of the fine line bundles 44 to each other in the intersecting regions 48 of the fine line bundles 44 and the welding of the linear members 52 to the braid tube 38 in the intersecting regions 48, together in one step. Thus, in the insertion tube for the endoscope of the present embodiment, the manufacturing efficiency of the insertion tube for the endoscope is improved.

In the above-mentioned embodiment, the linear members 52 are welded to the braid tube 38 in the respective intersecting regions 48 through which the linear members 52 pass, but welding portions may appropriately be selected from a number of intersecting regions 48 in accordance with desired resiliency. Moreover, the welding is used in restricting the linear members 52 to the braid tube 38, but bonding, brazing, soldering or the like may be used. Furthermore, the number of the linear members 52 may appropriately be set in accordance with the desired resiliency. For example, the eight linear members 52 may be arranged at an equal interval, that is, an interval of 45° in the peripheral direction of the braid tube 38.

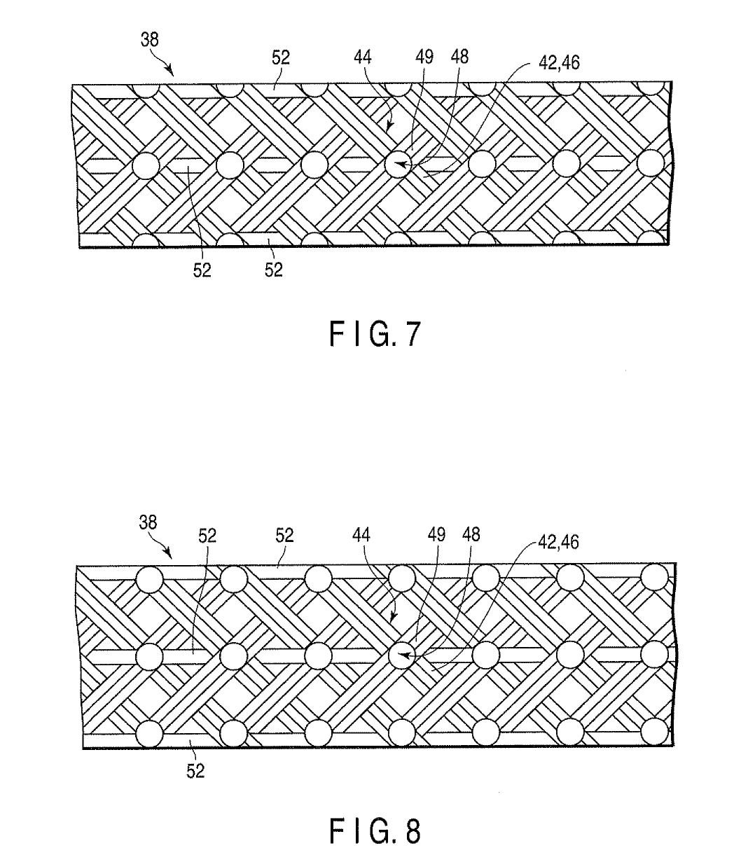

FIG. 7 shows a first modification example of the second embodiment of the present invention.

In the present modification example, the linear members 52 extend along the inner peripheral surface of the braid tube 38 in the longitudinal direction of the braid tube 38. Moreover, in the same manner as in the second embodiment, the linear members 52 are welded and restricted with the braid tube 38 in the intersecting regions 48 of the fine line bundles 44 of the braid tube 38.

FIG. 8 shows a second modification example of the second embodiment of the present invention.

In the present modification example, the linear members 52 are woven into the fine line bundles 44 of the braid tube 38, and extend in the longitudinal direction of the braid tube 38. Moreover, in the same manner as in the second embodiment, the linear members 52 are welded and restricted with the braid tube 38 in the intersecting regions 48 of the fine line bundles 44 of the braid tube 38.

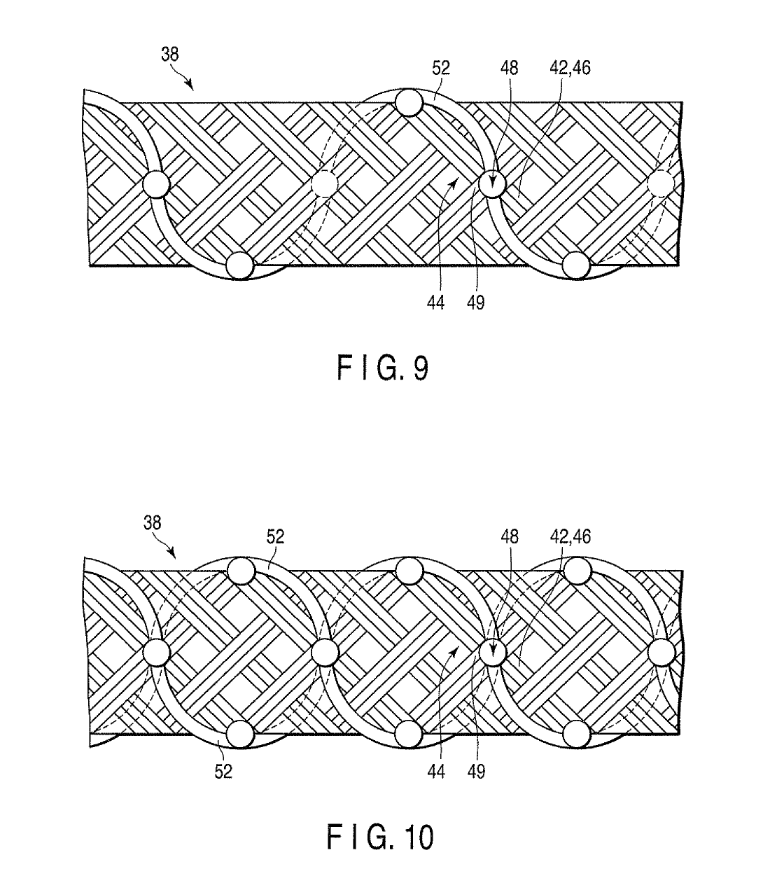

FIG. 9 shows a third embodiment of the present invention.

In the present embodiment, one linear member 52 spirally extends along the outer peripheral surface of the braid tube 38 in the longitudinal direction of the braid tube 38. Moreover, the linear member 52 is welded and restricted with the braid tube 38 in some intersecting regions 48 of the respective intersecting regions 48 of the fine line bundles 44 of the braid tube 38. Since the linear member 52 spirally extends in this manner, it is prevented that the softness of the insertion tube for the endoscope lowers owing to the linear member 52.

FIG. 10 shows a first modification example of the third embodiment of the present invention.

In the present modification example, two linear members 52 extend spirally in the same winding direction along the outer peripheral surface of the braid tube 38 in the longitudinal direction of the braid tube 38.

FIG. 11 shows a second modification example of the third embodiment of the present invention.

In the present modification example, two linear members 52 extend spirally in winding directions reverse to each other along the outer peripheral surface of the braid tube 38 in the longitudinal direction of the braid tube 38.

The above linear members 52 may be applied to the braid tube 38 formed of the chain link line netting or the expanded metal as described in the first or second modification example of the first embodiment, to improve the resiliency of the insertion tube for the endoscope.

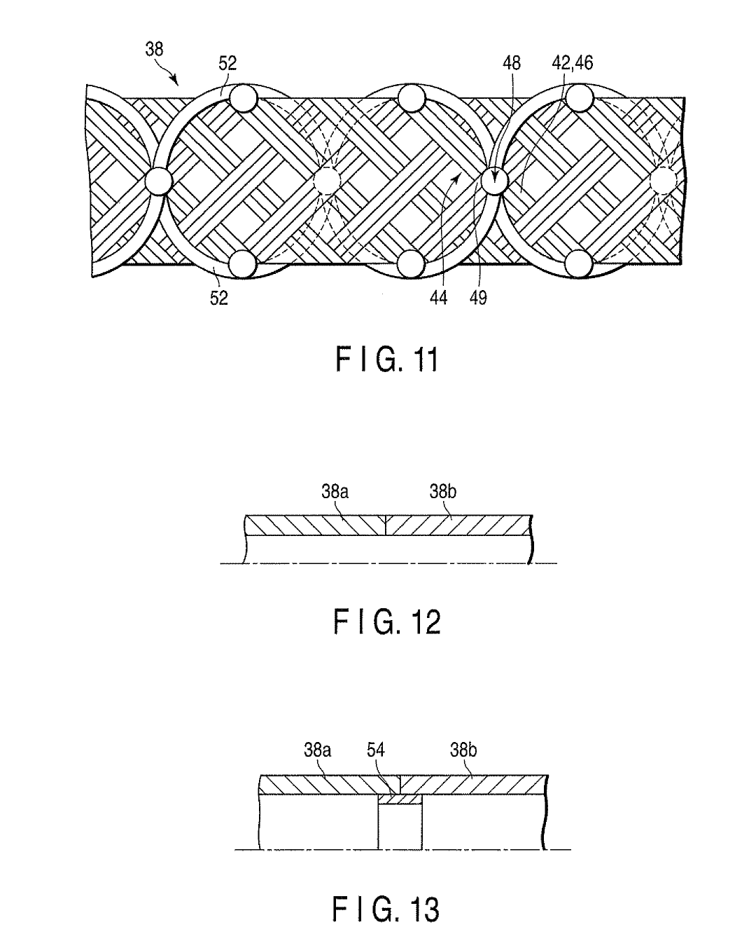

FIG. 12 shows a fourth embodiment of the present invention.

The braid tube 38 of the present embodiment is formed of a distal end braid tube 38a and a proximal end braid tube 38b. The distal end braid tube 38a is formed of metal fine lines made of a nickel-titanium alloy, and the proximal end braid tube 38b is formed of metal fine lines made of a stainless steel. Here, the nickel-titanium alloy has resiliency more excellent than that of the stainless steel, and the distal end braid tube 38a has the resiliency more excellent than that of the proximal end braid tube 38b. Therefore, the resiliency on the distal end side of the insertion tube for the endoscope is more excellent than that on the proximal end side thereof. The proximal end face of the distal end braid tube 38a and the distal end face of the proximal end braid tube 38b are abut on each other and joined together by welding, bonding, brazing, soldering or the like.

Thus, materials forming the portions of the braid tube 38 are different from each other, and so the characteristics of the portions of the braid tube 38 are different from each other, and therefore it is possible to improve the insertion and operation performance of the insertion tube for the endoscope.

FIG. 13 shows a first modification example of the fourth embodiment of the present invention.

In the present modification example, a connection tube 54 is fitted into the inside of the abutment portion between the proximal end face of the distal end braid tube 38a and the distal end face of the proximal end braid tube 38b, and the proximal end portion of the distal end braid tube 38a and the distal end portion of the proximal end braid tube 38b are joined to the outer peripheral surface of the connection tube 54.

Moreover, the connection tube 54 may be fitted onto the outside of the abutment portion between the proximal end face of the distal end braid tube 38a and the distal end face of the proximal end braid tube 38b, and the proximal end portion of the distal end braid tube 38a and the distal end portion of the proximal end braid tube 38b may be joined to the inner peripheral surface of the connection tube 54.

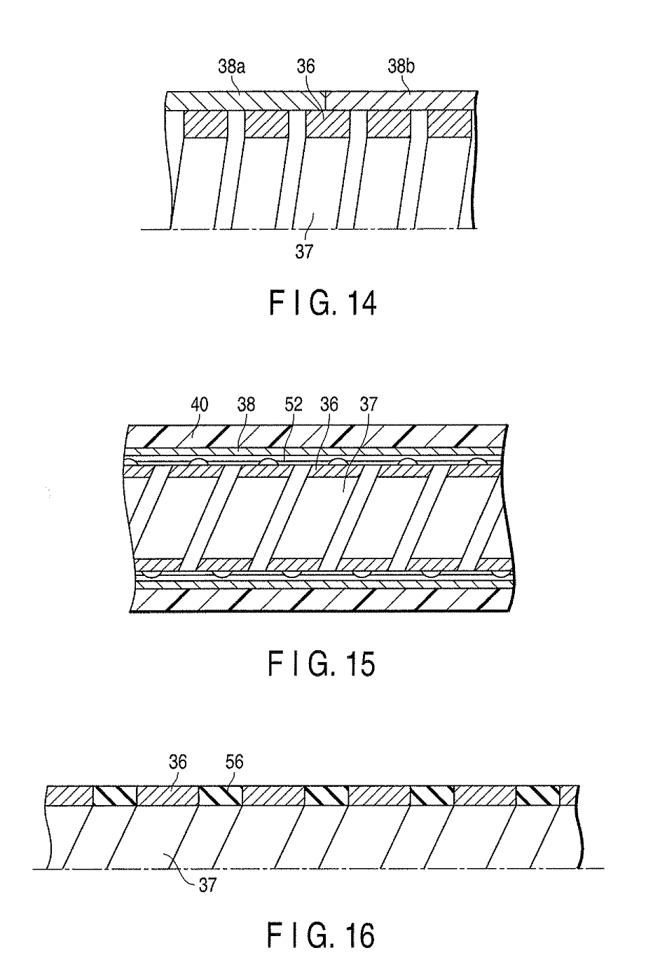

FIG. 14 shows a second modification example of the fourth embodiment of the present invention.

In the present modification example, a loop of the strip-like member 37 of the spiral tube 36 is arranged on the inside of the abutment portion of the proximal end face of the distal end braid tube 38a and the distal end face of the proximal end braid tube 38b, and the proximal end portion of the distal end braid tube 38a and the distal end portion of the proximal end braid tube 38b are joined to the outer peripheral surface of the strip-like member 37.

A fifth embodiment of the present invention will hereinafter be described.

In the present embodiment, the spiral tube 36 and the braid tube 38 are formed of a material having very excellent superelasticity (hereinafter referred to as the extrasuperelasticity). Examples of a material having the extrasuperelasticity include a superelastic plastic-type titanium alloy. The superelastic plastic-type titanium alloy is a β-type titanium alloy having a composition represented by Ti₃(Nb, Ta, V)+(Zr, Hf)+0, and it is possible to form a member having low Young's modulus and high strength through cold processing of the β-type titanium alloy.

Thus, the spiral tube 36 and the braid tube 33 are formed of the material having the extrasuperelasticity, and so the spiral tube 36 and the braid tube 38 includes the low Young's modulus and the high strength, and therefore it is possible to improve the softness, flexibility, resiliency and followability of the insertion tube for the endoscope to improve the insertion and operation performance of the insertion portion 20.

It is to be noted that instead of the spiral tube 36, a tube having the extrasuperelasticity may be used.

Reference embodiments of the present invention will hereinafter be described.

In the reference embodiments, the flexibility and resiliency of the insertion tube for the endoscope is improved by imparting excellent flexibility and resiliency to the spiral tube 36.

FIG. 15 shows a first reference embodiment of the present invention.

In the present reference embodiment, the linear member 52 similar to that of the second embodiment extends along the outer peripheral surface of the spiral tube 36 in the longitudinal direction of the spiral tube 36, and the linear member is joined to the outer peripheral surface of the spiral tube 36 in each loop of the strip-like member 37 of the spiral tube 36. As a joining method, welding, bonding, brazing, soldering or the like is used.

FIG. 16 shows a second reference embodiment of the present invention.

In the present reference embodiment, an elastic material 56 is interposed between both adjacent loops of the strip-like member 37 of the spiral tube 36. As the elastic material 56, thermoplastic elastomer, synthetic rubber or the like is used, and the elastic material is attached to the spiral tube 36 by welding, bonding or the like.

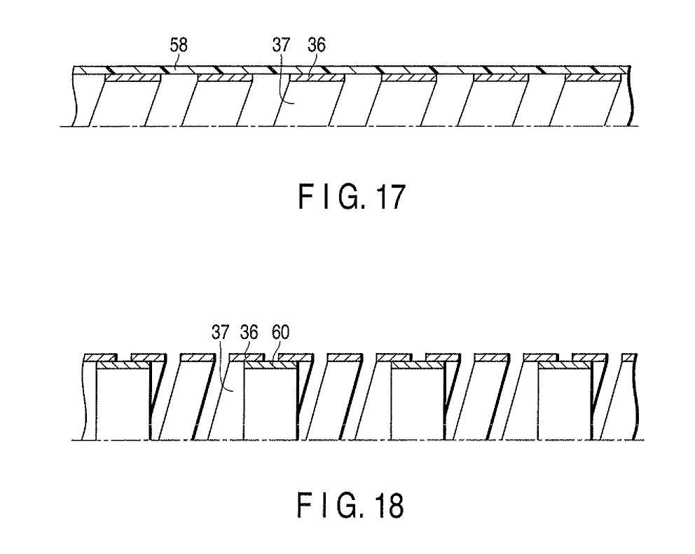

FIG. 17 shows a third reference embodiment of the present invention.

In the present reference embodiment, the spiral tube 36 is covered with an extremely thin tube 58 having elasticity. The tube 58 is formed of thermoplastic elastomer, synthetic rubber or the like.

FIG. 18 shows a fourth reference embodiment of the present invention.

In the present reference embodiment, a pipe 60 is fitted into the spiral tube 36. The pipe 60 is made of a resin or a metal, and has an axial length substantially equal to the pitch of the spiral tube 36. Both adjacent loops of the strip-like member 37 of the spiral tube 36 are joined to one end side portion and the other end side portion of the outer peripheral surface of the pipe 60, respectively, and the both adjacent loops are connected to each other. As a joining method, welding, bonding, brazing, soldering or the like is used.

In the above reference embodiments, since the spiral tube has sufficient flexibility and resiliency, the braid tube or the outer tube does not have to have such flexibility or resiliency. Therefore, as to the braid tube and the outer tube, the degree of freedom in material selection and design are increased. For example, it is possible to make the outer tube thinned to decrease the inner diameter or the outer diameter of the insertion tube for the endoscope. Moreover, as a material of the outer tube, a material which is comparatively soft but which has high resistance to chemicals may be used.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion tube for an endoscope comprising a braid tube, wherein the braid tube includes fine line portions arranged in a braided manner and intersecting portions where the fine line portions intersect with each other, and the fine line portions is restricted with each other in the intersecting portions.

2. The insertion tube for the endoscope according to claim 1, further comprising a linear member having superelasticity, extending from one end side to other end side of the braid tube, and restricted with the braid tube in the intersecting portions.

3. The insertion tube for the endoscope according to claim 1, wherein the braid tube is formed of braided fine line members, and the intersecting portions of the fine line members are welded to each other.

4. The insertion tube for the endoscope according to claim 1, wherein the braid tube is a chain link line netting.

5. The insertion tube for the endoscope according to claim 1, wherein the braid tube is an expanded metal.

6. The insertion tube for the endoscope according to claim 2, wherein the linear member is welded to the intersecting portions.

7. The insertion tube for the endoscope according to claim 2, wherein the linear member spirally extends in the longitudinal direction of the braid tube.

8. The insertion tube for the endoscope according to claim 1, wherein portions of the braid tube are made of materials which are different from each other.

9. The insertion tube for the endoscope according to claim 1, wherein the braid tube is made of a material having superelasticity.

10. An endoscope comprising an insertion tube for the endoscope, wherein the insertion tube for the endoscope includes a braid tube, the braid tube includes fine line portions arranged in a braided manner and intersecting portions where the fine line portions intersect with each other, and the fine line portions are restricted with each other in the intersecting portions.
